(19) United States
(12) Patent Application Publication
(10) Pub. No.: US 2022/0152239 A1
(43) Pub. Date: May 19, 2022

(54) Multi-Band Multi-Source Germicidal Lighting Apparatus

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/499,775

(22) Filed: Oct. 12, 2021

Related U.S. Application Data
(63) Continuation-in-part of application No. 17/140,673, filed on Jan. 4, 2021.
Application No. 17/140,673 is a continuation-in-part of application No. 17/137,763, filed on Dec. 30, 2020.
Application No. 17/137,763 is a continuation-in-part of application No. 17/099,271, filed on Nov. 16, 2020.

(51) Int. Cl. A61L 2/10; G06F 3/0346

(52) U.S. Cl. CPC A61L 2/10; A61L 2202/14; G06F 3/0346

(57) ABSTRACT

A multi-band multi-source germicidal lighting apparatus includes a first light source, a second light source, a first driver, a second driver, and a controller. The first light source emits a light in a 190˜280 nm wavelength range whereas the second light source emits a light in a 315˜420 nm wavelength range. The first driver is configured to convert an external power to an internal power to activate the first light source, and the second driver is configured to convert an external power to an internal power to activate the second light source, and the controller is configured to toggle the operation of the apparatus between a safe sanitation mode and a full sanitation mode. Optionally, a third light source emitting a visible light and a third driver are used for providing general lighting during the safe sanitation mode.

### CROSS REFERENCE TO RELATED PATENT APPLICATIONS

The present disclosure is part of a Continuation-in-Part (CIP) of US Patent application Ser. No. 17/140,673, filed 4 Jan. 2021, which is part of a CIP of U.S. patent application Ser. No. 17/137,763, filed 30 Dec. 2020, which is a CIP of U.S. patent application Ser. No. 17/099,271, filed 16 Nov. 2020, the contents of which being incorporated by reference in their entirety.

### BACKGROUND

### Technical Field

The present disclosure pertains to germicidal lighting devices and, more specially, proposes a multi-band multi-source germicidal lighting apparatus.

### Description Of Related Art

In the U.S. patent application Ser. No. 17/140,673, a multi-band germicidal irradiation apparatus was introduced. It includes a radiation source, a driver, and an optical filter. The radiation source emits wavelengths in three wavelength bands, 190˜230 nm, 230˜315 nm, and 315˜700 nm. The optical filter is a band-stop filter that filters the wavelength in a wavelength range of 230˜315 nm and permits the wavelength in wavelength ranges of 190˜230 nm and 315˜700 nm to pass through. It has the benefit of using both the far ultraviolet C (UVC) wavelength (190˜230 nm) and the ultraviolet A (UVA) wavelength (315˜400 nm) to achieve a higher antimicrobial effect. However, due to the limitation of having only one radiation source, it is therefore not possible to emit only the far UVC wavelength or the UVA wavelength, nor it is possible to emit the visible light (400˜700 nm) without emitting either far UVC or UVA wavelength.

The present disclosure proposes a multi-band multi-source germicidal lighting apparatus that uses multiple light sources and each with its own non-overlap wavelength band such that these light sources can be turned on individually or in combination as needed for achieving a greater operation flexibility for different operation scenarios.

### SUMMARY

In one aspect, the multi-band multi-source germicidal lighting apparatus comprises a first light source emitting a light in a 190˜280 nm wavelength range, a second light source emitting a light in a 315˜420 nm wavelength range, a first driver, a second driver, and a controller. The first driver is configured to convert an external power to an internal power to activate the first light source, and the second driver is configured to convert an external power to an internal power to activate the second light source. The controller is configured to turn on the first light source and the second light source individually or simultaneously. The wavelength range 315˜420 nm can be subdivided into the UVA wavelength range (315˜400 nm) and the near-UV wavelength range (400˜420 nm). Both have similar effect of damaging the cellular wall of bacteria by inducing reactive oxygen spices in the cellular wall. Their key difference lies in that the 315˜400 nm wavelength is invisible, which may be preferrable over the visible 400˜420 nm wavelength. Using a violet light in the 400˜420 nm wavelength range may alter the color distribution of the apparatus.

In some embodiments, the controller is configured to support two operation modes, namely the safe sanitation mode and the full sanitation mode. In the safe sanitation mode, the apparatus dispenses over a prorated 8-hour period an irradiation dosage less than the ACGIH (American Conference of Governmental Industrial Hygienists) specified Threshold Limit Values (TLVs) to the substance or surface to be disinfected by the apparatus. In the full sanitation mode, the apparatus dispenses over a prorated 8-hour period an irradiation dosage more than twice the ACGIH TLVs to the substance or surface to be disinfected by the apparatus. The ACGIH TLVs are shown in FIG. 1 (ACGIH ISBN: 0-9367-12-99-6). The safe sanitation mode is meant to be used when occupants are in the space and the full sanitation mode is meant for off-hours operation when nobody in the space. There are different configuration of the apparatus to support the safe sanitation mode. For example, by turning on only the second light source, or turning on both the first and the second light sources but at a lower irradiation, so long as the dispensed UV dosage prorated over 8 hours to the substance or surface to be disinfected is less than the ACGIH TLVs. Note that the controller may be configured to operate the apparatus in the safe mode less than 8 hours, e.g., for only 1 hours. However, so long the prorated dosage over 8 hours is less than ACGIH TLVs, it is considered safe by the ACGIH guidelines. Similarly, there are different configuration of the apparatus to support the full sanitation mode. For example, by turning on only the first light source, or by turning on both the first and the second light sources, so long as the dispensed UV dosage prorated over 8 hours to the substance or surface to be disinfected is more than twice the ACGIH TLVs.

There are different means for triggering the controller to switch between the safe sanitation mode and the full sanitation mode. In some embodiments, the controller is configured to operate according to an operation schedule to toggle between the safe sanitation mode and the full sanitation mode. The operation schedule may be stored locally in the controller or remotely on a scheduling device or a lighting control system.

In some embodiments, the apparatus further comprises a motion sensor working in conjunction with the controller such that when a motion is detected, the controller is configured to operate the apparatus in the safe sanitation mode, and when no motion is detected, the controller is configured to operate the apparatus in the full sanitation mode. It is also possible for a controller to take into the account of both an operation schedule and the motion detection in setting the operation mode. FIG. 5 shows an example of such operation configuration. During 7:00 am to 7:00 pm, the controller operates the apparatus in the safe sanitation mode. Then from 7:00 pm to 7:am, the controller operates the apparatus in full sanitation mode by default. However, upon the defecting of motion, the controller will change temporarily to the safe sanitation mode, and later resumes the full sanitation mode when no further motion is detected in the space.

In some embodiments, the first light source may have a peak wavelength in the range of 190˜230 nm, i.e., it is a far UVC light source. It is known that the far UVC wavelength doesn't penetrate the skin as deep as the regular UVC wavelength does. Thus, it may be assumed that the far UVC photon energy may be weakened when passing through the cellular wall of bacteria before striking the bacterial DNA/RNA structures. Since the UVA (or near-UV 405 nm) wavelength is known to damage the cellular wall of bacteria by triggering reactive oxygen spices in bacterial cellular wall, it would make sense to turn on both the first light source in 190˜230 nm wavelength range and the second light source in 315˜4200 nm wavelength range during the full sanitation mode. This could benefit from the UVA wavelength damaging the bacterial cellular wall and thus making it easier for the far UVC photons to pass through the damaged cellular wall without losing too much photon energy, resulting in more damages to the DNA/RNA structures of the bacteria and achieving a more effective germicidal irradiation. This argument could be applied to the lipid-enveloped viruses as well since their viral envelope forms a membrane and is susceptible to similar reactive oxygen spices damage by the irradiation of the 315˜420 nm wavelength.

In another aspect, the multi-band multi-source germicidal lighting apparatus comprises a first light source emitting a light in a 190˜280 nm wavelength range, a second light source emitting a light in a 315˜420 nm wavelength range, a third light source emitting primarily a visible light for general illumination, a first driver, a second driver, a third driver, and a controller. The first driver is configured to convert an external power to an internal power to activate the first light source. The second driver is configured to convert an external power to an internal power to activate the second light source. The third driver is configured to convert an external power to an internal power to activate the third light source The controller is configured to turn on any combination of the first light source, the second light source, and the third light source.

In some embodiments, the controller is configured to support at least two operation modes, the safe sanitation mode and the full sanitation mode. In the safe sanitation mode, the apparatus dispenses over a prorated 8-hour period an irradiation dosage less than the ACGIH TLVs to the substance or surface to be disinfected by the apparatus. In the full sanitation mode, the apparatus dispenses over a prorated 8-hour period an irradiation dosage more than twice the ACGIH TLVs to the substance or surface to be disinfected by the apparatus. The controller may support a third mode, which is a non-sanitation mode. Under the non-sanitation mode, the controller would only turn on the third light source and keep the first and the second light sources off.

In some embodiments, the controller is configured to operate according to an operation schedule to toggle between the safe sanitation mode and the full sanitation mode. The operation schedule may be stored locally in the controller or remotely on a scheduling device or a lighting control system.

In some embodiments, the apparatus further comprises a motion sensor working in conjunction with the controller such that when a motion is detected, the controller is configured to operate the apparatus in the safe sanitation mode, and when no motion is detected, the controller is configured to operate the apparatus in the full sanitation mode.

In some embodiments, the first light source may have a peak wavelength in the range of 190˜230 nm, i.e., it is a far UVC light source.

It is feasible to use two different germicidal lighting devices, one device emitting the UVC light and the other device emitting the UVA light, and a separate motion sensor to control which of the two devices will be turned on based on motion detection. While such system configuration is not a standalone apparatus per se, it nonetheless employs the same principal concept of the present disclosure. Therefore, in another aspect of the present disclosure, a multi-band multi-source germicidal lighting method may have a first light source emitting a light in a 190˜280 nm wavelength range, a second light source emitting a light in a 315˜420 nm wavelength range, and a controlling mechanism. The controlling mechanism may be configured to support two operation modes, the safe sanitation mode and the full sanitation mode. In the safe sanitation mode, the method dispenses over a prorated 8-hour period an irradiation dosage less than the ACGIH TLVs to the substance or surface to be disinfected by the method. In the full sanitation mode, the method dispenses over a prorated 8-hour period an irradiation dosage more than twice the ACGIH TLVs to the substance or surface to be disinfected by the method. The controlling mechanism may be a physical controller hardware, or it may be a software program on an app or a lighting control system.

In some embodiments, the controlling mechanism operates according to an operation schedule to toggle between the safe sanitation mode and the full sanitation mode.

In some embodiments, the method may further include a motion sensing mechanism such that when a motion is detected, the controlling mechanism is configured to operate in the safe sanitation mode, and when no motion is detected, the controlling mechanism is configured to operate in the full sanitation mode.

In some embodiments, the first light source may have a peak wavelength in the range of 190˜230 nm, i.e., it is a far UVC light source.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

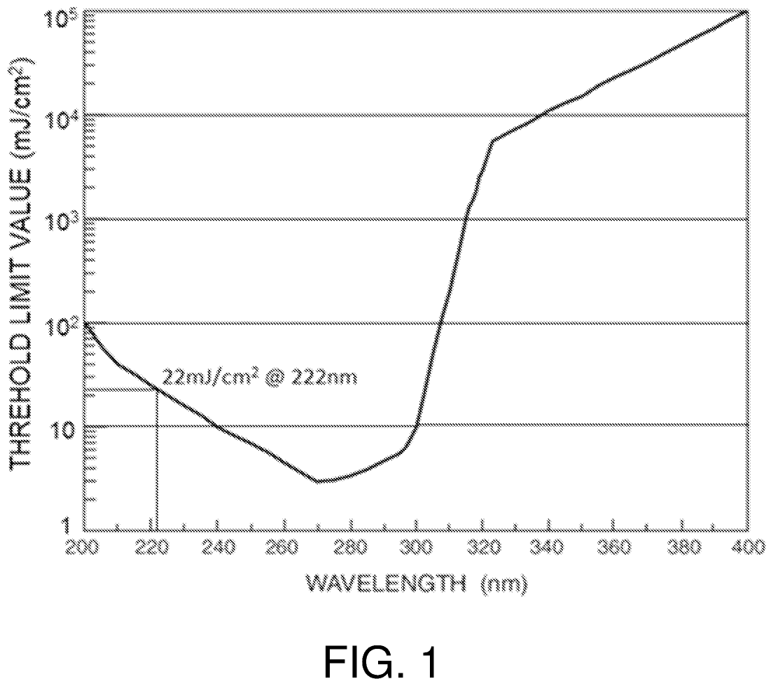

FIG. 2 schematically depicts a diagram of an embodiment of the present disclosure with two light sources.

FIG. 3 schematically depicts a diagram of an embodiment of the present disclosure with three light sources.

FIG. 4 schematically depicts a diagram of an embodiment of the present disclosure with three light sources and a motion sensor.

FIG. 5 shows an operation schedule example.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of multi-band multi-source germicidal lighting apparatuses having different form factors.

The present disclosure includes a first light source, a second light source, a first driver, a second driver, and a controller. The first light source emitting a light in a 190˜280 nm wavelength range whereas the second light source emitting a light in a 315˜420 nm wavelength range. The first driver is configured to convert an external power to an internal power to activate the first light source, and the second driver is configured to convert an external power to an internal power to activate the second light source, and the controller is configured to toggle the operation of the apparatus between a safe sanitation mode and a full sanitation mode. In some embodiment a third light source emitting a visible light and a third driver are used for providing general lighting during the safe sanitation mode.

### EXAMPLE IMPLEMENTATIONS

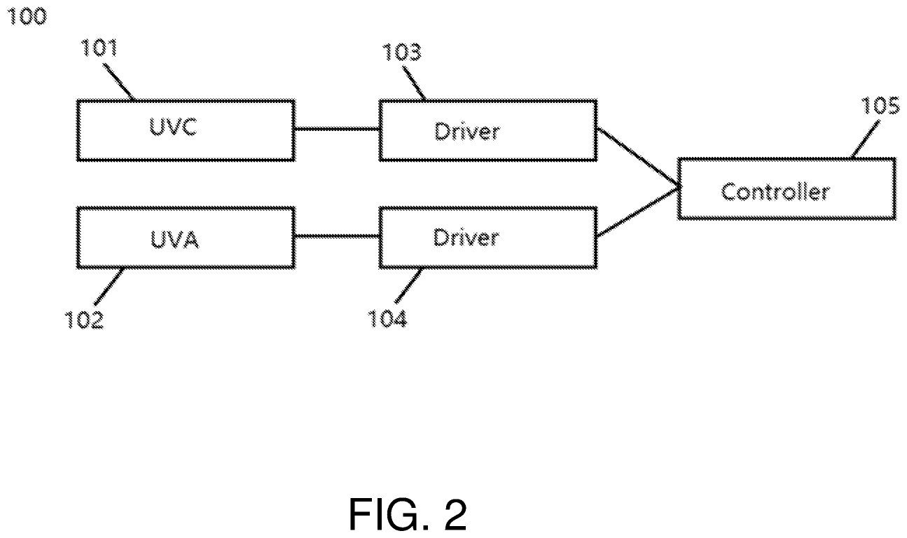

FIG. 2 is an embodiment of the multi-band multi-source germicidal lighting apparatus of the present disclosure. The apparatus 100 includes a UVC light source 101, a UVA light source 102, a first driver 103, a second driver 104, and a controller 105. The first driver 103 converts an external power to an internal power for activating the UVC light source 101, and the second driver 104 converts an external power to an internal power for activating the UVA light source 102. The controller 105 can turn on either the UVA light source 102 for the safe sanitation mode operation, or the UVC light source 101 for the full sanitation mode operation. If this embodiment is to be installed in a ceiling, then it may be assumed that its shortest distance to an occupant would approximately 4-ft feet. In the safe sanitation mode, the pre-configured UV dosage of this embodiment to a substance or surface at a 4-feet distance would be less than the ACGIH TLVs over a prorated 8-hour period. In the full sanitation mode, the pre-configured UV dosage of this embodiment to a substance or surface at a 4-feet distance would be more than twice the ACGIH TLVs over a prorated 8-hour period. It is foreseeable that a more advanced embodiment may allow a user to adjust the UV dosage according to the mounting height of the embodiment (subsequently, the closest distance to the occupant in the space), through either a configurable controller 105 or adjustable drivers 103, 104, or both.

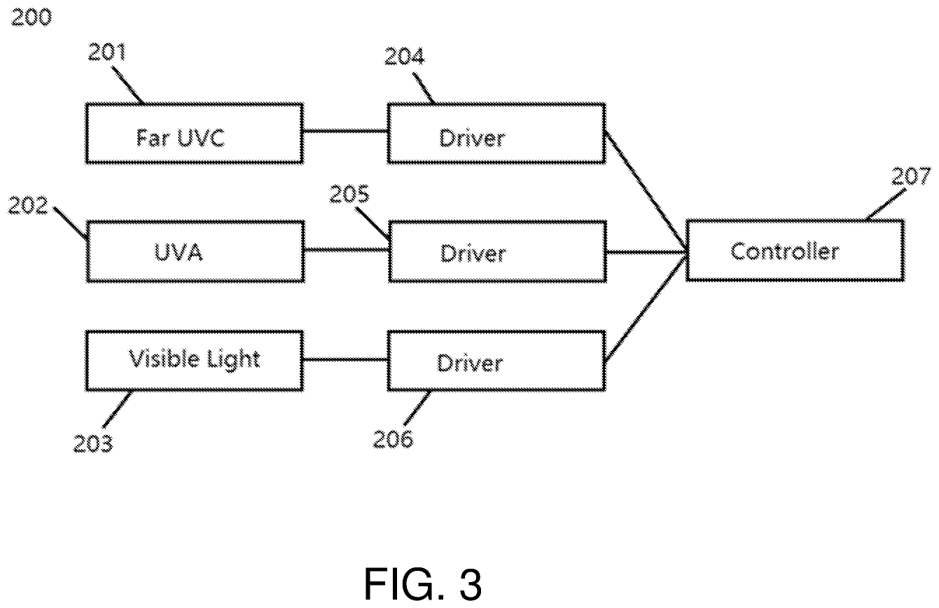

FIG. 3 is another embodiment of the present disclosure 200. The apparatus 200 includes a far UVC light source 201, a UVA light source 202, a visible light source 203, a first driver 204, a second driver 205, a third driver 206, and a controller 207. During the safe sanitation mode, the controller 207 will turn on both the visible light source 203 and the UVA light source 202. During the full sanitation mode, the controller 207 will turn on both the far UVC light source 201 and the UVA light source 202. The far UVC light source 201 has a peak wavelength at 222 nm. The UV dosage of the embodiment may be configured according to the intended mounting height of the embodiment.

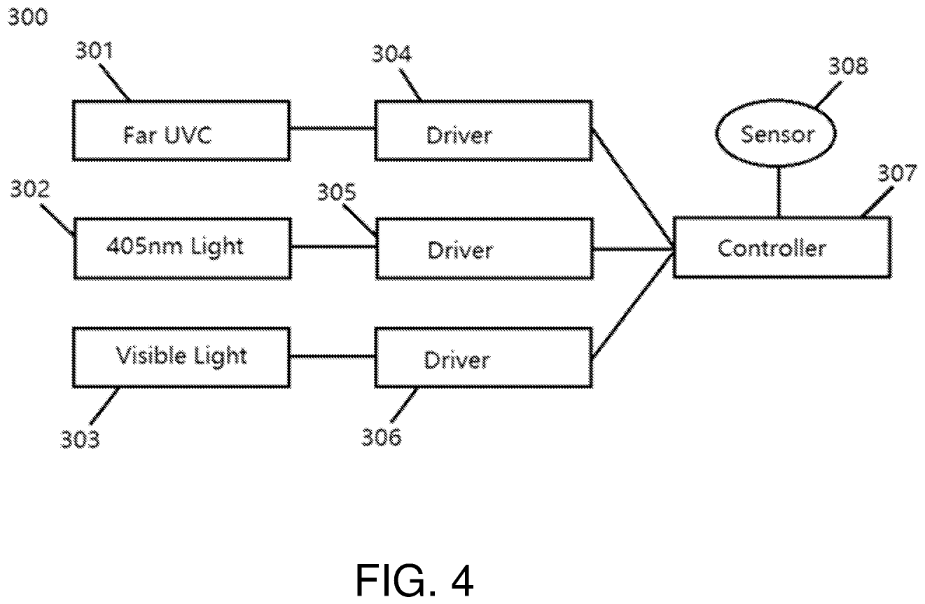

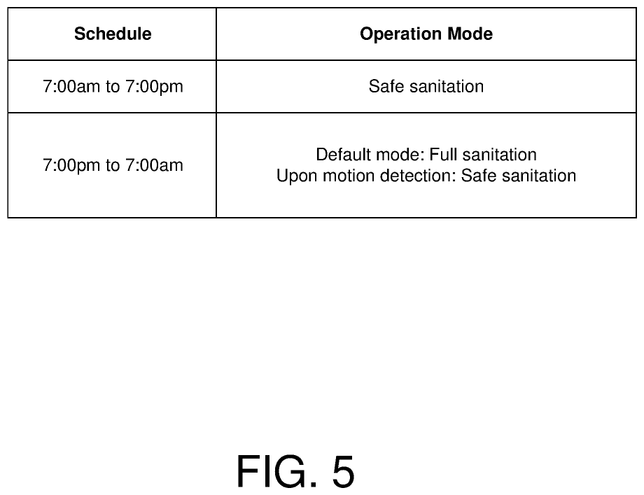

FIG. 4 is yet another embodiment of the present disclosure 300. The apparatus 300 includes a far UVC light source 301, a 405 nm light source 302, a visible light source 303, a first driver 304, a second driver 305, a third driver 306, a controller 307, and a motion sensor 308. During the safe sanitation mode, the controller 307 will turn on both the visible light source 303 and the 405 nm light source 302. The visible light source 303 provides general lighting for the space during the safe sanitation mode. During the full sanitation mode, the controller 307 will turn on both the far UVC light source 301 and the 405 nm light source 302. FIG. 5 shows an operation schedule example for the embodiment 300. During 7:00 am to 7:00 pm, the controller 307 will operate this embodiment in the safe sanitation mode, regardless of the motion detection. During 7:00 pm to 7:00 am, the controller will operate in the full sanitation mode by default. However, if a motion is detected by the motion sensor 308 during this period, the controller will switch the operation to the safe sanitation mode temporarily, avoiding any occupant from being over-exposed to the UV light. When no further motions are detected by the motion sensor, the controller will resume the operation to the full sanitation mode. Note that the 405 nm light source 302 used in this embodiment may emit a noticeable visible violet light during the safe sanitation mode. It is recommended to set the light output level of the 405 nm light source 302 to be much smaller than the light output level of the visible light source 303 in order to minimize the color temperature shift of this embodiment during the safe sanitation mode. This would not be an issue during the full sanitation mode since there are not occupants in the space. It is foreseeable to implement an embodiment that could crank up the irradiation of the 405 nm light source 302 during the full sanitation mode for a better antimicrobial effect.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A germicidal lighting apparatus, comprising:
a first light source configured to emit a light in a wavelength range of 190˜280 nm;
a second light source configured to emit a light in a wavelength range of 315˜420 nm;
a first driver;
a second driver; and
a controller,
wherein:
the first driver is configured to convert an external power to a first internal power to activate the first light source,
the second driver is configured to convert the external power to a second internal power to activate the second light source, and
the controller is configured to turn on the first light source and the second light source individually or simultaneously.

2. The apparatus of claim 1, wherein the controller is configured to support two operation modes comprising a safe sanitation mode and a full sanitation mode such that:
in the safe sanitation mode, the apparatus dispenses over a prorated 8-hour period an irradiation dosage less than American Conference of Governmental Industrial Hygienists (ACGIH)-specified Threshold Limit Values (TLVs) to a substance or surface to be disinfected by the apparatus; and
in the full sanitation mode, the apparatus dispenses over a prorated 8-hour period an irradiation dosage more than twice the ACGIH-specified TLVs to the substance or surface to be disinfected by the apparatus.

3. The apparatus of claim 2, wherein the controller is configured to operate according to an operation schedule to toggle between the safe sanitation mode and the full sanitation mode.

4. The apparatus of claim 2, further comprising:
a motion sensor coupled to and working in conjunction with the controller, wherein:
when a motion is detected by the motion sensor, the controller is configured to operate the apparatus in the safe sanitation mode, and
when no motion is detected by the motion sensor, the controller is configured to operate the apparatus in the full sanitation mode.

5. The apparatus of claim 1, wherein the first light source has a peak wavelength in a wavelength range of 190˜230 nm.

6. A germicidal lighting apparatus, comprising:
a first light source configured to emit a light in a wavelength range of 190˜280 nm;
a second light source configured to emit a light in a wavelength range of 315˜420 nm;
a third light source configured to emit a visible light as general illumination;
a first driver;
a second driver;
a third driver; and
a controller,
wherein:
the first driver is configured to convert an external power to a first internal power to activate the first light source,
the second driver is configured to convert the external power to a second internal power to activate the second light source,
the third driver is configured to convert the external power to a third internal power to activate the third light source, and
the controller is configured to turn on any combination of the first light source, the second light source, and the third light source.

7. The apparatus of claim 6, wherein the controller is configured to support at least two operation modes comprising a safe sanitation mode and a full sanitation mode such that:
in the safe sanitation mode, the apparatus dispenses over a prorated 8-hour period an irradiation dosage less than American Conference of Governmental Industrial Hygienists (ACGIH)-specified TLVs to a substance or surface to be disinfected by the apparatus; and
in the full sanitation mode, the apparatus dispenses over a prorated 8-hour period an irradiation dosage more than twice the ACGIH-specified TLVs to the substance or surface to be disinfected by the apparatus.

8. The apparatus of claim 7, wherein the controller is configured to operate according to an operation schedule to toggle between the safe sanitation mode and the full sanitation mode.

9. The apparatus of claim 7, further comprising:
a motion sensor coupled to and working in conjunction with the controller, wherein:
when a motion is detected by the motion sensor, the controller is configured to operate the apparatus in the safe sanitation mode, and
when no motion is detected by the motion sensor, the controller is configured to operate the apparatus in the full sanitation mode.

10. The apparatus of claim 6, wherein the first light source has a peak wavelength in a wavelength range of 190˜230 nm.

11. A germicidal lighting apparatus, having:
a first light source configured to emit a light in a wavelength range of 190˜280 nm;
a second light source configured to emit a light in a wavelength range of 315˜420 nm; and
a controlling mechanism,
wherein the controlling mechanism is configured to support two operation modes comprising a safe sanitation mode and a full sanitation mode such that:
in the safe sanitation mode, the apparatus dispenses over a prorated 8-hour period an irradiation dosage less than American Conference of Governmental Industrial Hygienists (ACGIH)-specified TLVs to a substance or surface to be disinfected, and
in the full sanitation mode, the apparatus dispenses over a prorated 8-hour period an irradiation dosage more than twice the ACGIH-specified TLVs to the substance or surface to be disinfected.

12. The apparatus of claim 11, wherein the controlling mechanism operates according to an operation schedule to toggle between the safe sanitation mode and the full sanitation mode.

13. The apparatus of claim 11, further comprising:
a motion sensing mechanism coupled to the controlling mechanism, wherein:
when a motion is detected by the motion sensing mechanism, the controlling mechanism is configured to operate in the safe sanitation mode, and
when no motion is detected by the motion sensing mechanism, the controlling mechanism is configured to operate in the full sanitation mode.

14. The apparatus of claim 11, wherein the first light source has a peak wavelength in a wavelength range of 190˜230 nm.
